(19) United States
(12) Patent Application Publication
(10) Pub. No.: US 2005/0065219 A1
(43) Pub. Date: Mar. 24, 2005

(54) Treatment of demyelinating conditions

(76) Inventors: Stuart A. Lipton, Rancho Santa Fe, CA (US); Gregory T. Went, Mill Valley, CA (US); Laurence R. Meyerson, San Rafael, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/810,848

(22) Filed: Mar. 26, 2004

Related U.S. Application Data
(60) Provisional application No. 60/458,050, filed on Mar. 27, 2003.

(51) Int. Cl. A61K 31/13; A61K 31/40

(52) U.S. Cl. 514/662; 514/408

(57) ABSTRACT

The present invention provides novel methods and compositions for the treatment of multiple sclerosis.

### RELATED APPLICATIONS

This application claims priority to U.S. Ser. No. 60/458,050 filed Mar. 27, 2003 the contents of which is incorporated herein by reference in its entirety.

### FIELD OF THE INVENTION

This invention relates to compositions and methods comprising an uncompetitive NMDA receptor channel antagonist for treatment of demyelinating conditions, such as multiple sclerosis.

### BACKGROUND OF THE INVENTION

a) Indication Treated

Multiple sclerosis (MS) is a progressive central nervous system (CNS) disease that affects over 250,000 Americans. MS is characterized by neuron deterioration in the central nervous system with the associated loss of the insulating myelin sheath from around the axons of the nerve cells (demyelination). This loss of myelin results in loss of electrical insulation and the “short-circuiting” of the electrical pathways mediated by the affected nerves and progressive neurological impairment.

In multiple sclerosis patches of myelin are destroyed by the body's own immune system via a chronic inflammatory autoimmune reaction. This destruction leads to scarring and damage to the underlying nerve fibers, and may manifest itself in a variety of symptoms, depending on the parts of the brain and spinal cord that are affected.

The symptoms associated with MS include pain and tingling in the arms and legs; localized and generalized numbness, muscle spasm and weakness; bowel and bladder dysfunction; difficulty with balance when walking or standing; and fatigue. In most cases, people afflicted with MS lose the ability to stand and/or walk entirely. Optic neuritis may occur episodically throughout the course of the disease. The symptoms are exacerbated by physical fatigue or emotional stress.

Approximately half the people with this disease have relapsing-remitting MS in which there are unpredictable attacks where the clinical symptoms become worse (exacerbation) which are separated by periods of remission where the symptoms stabilize or diminish. The other half have chronic progressive MS without periods of remission.

When flare-ups and exacerbations in MS occur, patients are often treated with high doses of oral or intravenous steroids which may temporarily ameliorate some of the multiple sclerosis symptoms. The gradual nervous system deterioration persists despite this treatment.

Another condition for which there is a long felt need for a non-stimulant pharmacological therapy is the fatigue associated with multiple sclerosis (MS). In one study involving 656 patients with MS, 78% complained of fatigue, 60% experienced it every day, and 22% suffered disruption of their daily activities (Freal et al., Arch. Phys. Med. Rehabil. 65:135, 1984). The National Multiple Sclerosis Society evaluated 839 patients who had only minor neurologic impairment despite having had MS for longer than 10 years, and fatigue was the most commonly reported symptom in this group of mildly affected patients (Jones, New York: National multiple sclerosis Society, Health Services Research Report, 1991). In another study 40% of MS patients listed fatigue as the most serious symptom of their disease (Murray, Can. J. Neurol. Sci. 12:251, 1985). Fatigue is reported to be the cause of at least temporary disability in up to 75% of patients with MS.

b) Prior Uses of Uncompetitive NMDA Receptor Channel Antagonists in This and Related Indications

Amantadine has been used to treat MS related fatigue. Although the mechanism of MS fatigue is poorly understood it has been attributed to nerve conduction abnormalities within the central nervous system and increased energy demands caused by neurologic disability. Several characteristics of MS fatigue are interference with physical functioning and activities of daily living, aggravation by heat, and worsening at the end of the day. Medications that are prescribed for the treatment of MS fatigue include amantadine, pemoline, and other stimulants. Amantadine has been demonstrated to benefit MS fatigue in 79% of patients in a double blind, randomized study, but its mechanism of beneficial action is not known (Krupp et al., Neurology 45:1956, 1995). Although amantadine has been demonstrated in a rigorous fashion to benefit MS fatigue, the benefit is partial for most patients and there are still significant numbers of patients who report no benefit.

More generally, uncompetitive NMDA receptor channel antagonists like memantine (EBIXA™) are known to be neuroprotective, with their action being felt almost entirely on neurons in an excitotoxic state caused by elevated glutamate, the primary exicitatory neurotransmitter. Excessive glutamate can also lead to increased risk of neuronal apoptosis, which is thought to contribute to progress in MS and other neurodegenerative indications. Recently, the FDA has approved memantine (NAMENDA™) for use in treating Alzheimers Disease in the United States.

c) Prior Uses of Other Therapeutics in This Indication

Several general therapeutic approaches have been tried to limit the immune-mediated CNS damage in MS, including antigen-non-specific immunosuppressive drugs and treatments; antigen-specific immunosuppressive drugs and treatments; and cytokine-specific therapies. Some current monotherapies for multiple sclerosis include corticosteroid drugs such as methylprednisolone (SOLUMEDROL™) to alleviate the symptoms of acute episodes, muscle relaxants such as tizanidine hydrochloride (ZANAFLEX™), as well as other biomolecules such as glatiramer acetate (COPAXONE™), and mitoxantrone (NOVANTRONE™). In particular, β-interferons (IFN-β) have been tested and approved by the U.S. Food and Drug Administration (FDA) as an MS therapy, e.g., interferon-β1a (AVONEX™, REBIF™) or interferon-β1b (BETASERON™). Other drugs, e.g., τ-interferon (see, e.g., U.S. Pat. No. 6,060,450), vitamin D analogs, e.g., 1,25(OH)₂D₃ (see, e.g., U.S. Pat. No. 5,716,946), IFN-β-2 (U.S. Patent Publication No. 20020025304), spirogermaniums, (see, e.g., U.S. Pat. No. 4,654,333), prostaglandins, e.g., latanoprost, brimonidine, PGE1, PGE2 or PGE3. (see, e.g., U.S. Patent Publication No. 20020004525), tetracyclines and derivatives thereof, e.g., minocycline, doxycycline (U.S. Patent Publication No. 20020022608), are known.

### SUMMARY OF THE INVENTION

The present invention provides a method of treatment for multiple sclerosis, and pharmaceutical compositions for treating multiple sclerosis.

In an embodiment, the invention relates to methods for treating multiple sclerosis through the administration of one or more amino-adamantane-derived uncompetitive NMDA receptor channel antagonists, such as memantine, rimantadine, and amantadine. In this embodiment, an uncompetitive NMDA receptor channel antagonist is administered to a subject having multiple sclerosis, such that the multiple sclerosis is treated or at least partially alleviated. The uncompetitive NMDA receptor channel antagonists are administered as part of a pharmaceutical composition. In another embodiment, a patient is diagnosed, e.g., to determine if treatment is necessary, whereupon a therapy in accordance with the invention is administered to treat the patient.

In an embodiment, the invention relates to methods for treating symptoms associated with multiple sclerosis through the administration of one or more uncompetitive NMDA receptor channel antagonists, such as memantine, rimantadine, and amantadine. In this embodiment, a known uncompetitive NMDA receptor channel antagonist is administered to a subject having multiple sclerosis, such that the multiple sclerosis is treated or at least partially alleviated.

Symptoms associated with, or arising from, multiple sclerosis, including fatigue, pain and tingling in the arms and legs; localized and generalized numbness, muscle spasm and weakness; bowel and bladder dysfunction; and difficulty with balance when walking or standing. The amount of uncompetitive NMDA receptor channel antagonist and/or a multiple sclerosis agent is typically effective to reduce symptoms and to enable an observation of a reduction in symptoms.

The present invention also provides for compositions which include amino-adamantane-derived uncompetitive NMDA receptor channel antagonist agents, and are used in the treatment of patients suffering from multiple sclerosis.

In some embodiments, the uncompetitive NMDA receptor channel antagonist agents are administered as part of a pharmaceutical composition. In another embodiment, a patient is diagnosed, e.g., to determine if treatment is necessary, whereupon a pharmaceutical composition in accordance with the invention is administered to treat the patient. The amount of uncompetitive NMDA receptor channel antagonist agent is typically effective to reduce symptoms and to enable an observation of a reduction in symptoms.

Advantageously, the amino-adamantane-derived uncompetitive NMDA receptor channel antagonist agents which are used in the invention include memantine (1-amino-3,5-dimethyladamantane), rimantadine (1-(1-aminoethyl)adamantane), or amantadine (1-amino-adamantane). Other amino-adamantane-derived uncompetitive NMDA receptor channel antagonist agents are those described in U.S. Pat. No. 5,061,703.

Uncompetitive NMDA receptor channel antagonist agents are administered at a dosage of generally from 30-400 mg/day. For example, for memantine the dosage is preferably greater than 30 mg/day, e.g., about from about 30 to about 80 mg/day. Memantine is administered at 30, 40, 50, 60, 70, or 80 mg/day. Amantadine is administered from about 150 to about 400 mg/day, e.g., at 180, 200, 250, 300, 350, or 400 mg/day. Rimantadine is administered from about 150 to about 400 mg/day, e.g., at 180, 200, 250, 300, 350, or 400 mg/day. Memantine is particularly preferred.

Administration of the therapies of the invention may be orally, topically, intranasally, subcutaneously, intramuscularly, or intravenously.

The invention further relates to kits for treating patients having multiple sclerosis, comprising a therapeutically effective dose of an uncompetitive NMDA receptor channel antagonist, and instructions for its use.

Pharmaceutical compositions comprising an uncompetitive NMDA receptor channel antagonist, in an effective amount(s) to treat multiple sclerosis, are also included in the invention.

The above description sets forth rather broadly the more important features of the present invention in order that the detailed description thereof that follows may be understood, and in order that the present contributions to the art may be better appreciated. Other objects and features of the present invention will become apparent from the following detailed description.

### DETAILED DESCRIPTION OF THE INVENTION

The features and other details of the invention will now be more particularly described and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified. The scientific publications, patents or patent applications cited in the various sections of this document are herein incorporated-by-reference for all purposes.

Definitions

For convenience, certain terms used in the specification, examples, and appended claims are collected here.

As used herein, the term “Agent” includes a protein, polypeptide, peptide, nucleic acid (including DNA or RNA), antibody, molecule, compound, antibiotic, or drug, and any combinations thereof.

“Treating”, includes any effect, e.g., lessening, reducing, modulating, or eliminating, that results in the improvement of the condition, disease, disorder, etc.

Preferably, the term “Subject” refers to a mammal. More preferably, the term subject refers to a primate. More preferably, the term “subject” refers to a human.

“Multiple Sclerosis Symptoms,” includes the commonly observed symptoms of multiple sclerosis, such as those described in Treatment of Multiple Sclerosis: Trial Design, Results, and Future Perspectives, ed. Rudick and D. Goodkin, Springer-Verlag, New York, 1992, particularly those symptoms described on pages 48-52.

“Pharmaceutically or Pharmacologically Acceptable” include molecular entities and compositions that do not produce an adverse, allergic or other untoward reaction when administered to an animal, or a human, as appropriate.

“Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

“Pharmaceutically Acceptable Salts” include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

“Uncompetitive NMDA receptor channel antagonists” include amino-adamantanes, nitro-amino-adamantanes, nitrone-adamantanes, nitroxide-adamantanes, and derivatives thereof. Amino-adamantanes and derivatives include amino-adamantane derived or amantadine-derived molecules capable of acting as antagonists of the N-methyl-D-aspartate (NMDA) type receptors, and pharmaceutically acceptable salts and esters thereof. Members of the uncompetitive NMDA receptor channel antagonist family include those described in U.S. Pat. No. 5,061,703. Preferably, the uncompetitive NMDA receptor channel antagonists of the invention are amantadine, memantine, and rimantadine.

Preferred uncompetitive NMDA receptor channel antagonists have no active metabolites that possess NMDA antagonizing properties and have serum levels available for measurement.

Amino-adamantanes

Certain amino-adamantane, uncompetitive NMDA receptor channel antagonists have been used to treat illnesses. One uncompetitive NMDA receptor channel antagonist is memantine, which is currently approved for the treatment of Alzheimer's disease and for the treatment of Parkinson's associated spasticity in Germany (Schneider et al., Dtsch. Med. Wschr. 109:987 (1984)) and is under clinical investigation for the treatment of various neurodegenerative diseases. Recently, the FDA has approved memantine (EBIXA™ NAMENDA™) for use in treating Alzheimers Disease in the United States.

Uncompetitive NMDA receptor channel antagonists, like memantine, are known to be neuroprotective, their action being exerted almost entirely on neurons in an excitotoxic state caused by elevated glutamate levels and increases in cellular calcium concentrations. Glutamate is an important excitatory neurotransmitter. Excessive glutamate can also lead to increased risk of neuronal apoptosis, also which is thought to contribute to progression in neurodegeneration.

Without wishing to be bound by theory, it is thought that memantine exerts a neuroprotective effect because it is a micromolar antagonist of the NMDA receptor channel (Bormann J., Eur. J. Pharmacol. 166:59 1 (1989)). Memantine protects cortical and retinal neuron cultures from the toxicity of glutamate, NMDA and the HIV-1 coat protein gp120 (Deyer et al., Science 248:364, 1990). Memantine has antihypoxic properties in vitro and in vivo. Memantine also prevents quinolic acid-induced hippocampal damage in rats (Keilhoff et al., Eur. J. Pharmacol. 219:451, 1992). Although structurally quite different from other NMDA channel blockers, memantine inhibits [³H]dizocilpine (Chen et al., J. Neurosci. 12: 4427, 1992) binding to brain membranes. Memantine also blocks other neurotransmitter-gated ionotropic receptors, including nicotinic acetylcholine receptors (Masou et al., Eur. J. Pharmacol. 130: 187, 1986) and 5-hydroxytryptamine 5-HT₃ receptors (Reiser et al., Brain Res. 443: 338, 1988). Memantine demonstrates anti-hypoxic properties in vitro and in vivo.

Compared to the other NMDA antagonists, memantine has been reported to have the greatest effective potency for binding at the PCP and MK-801 receptor sites in human brain tissue (Kornhuber et al., Eur J Pharmacol (Mod Pharmacol Sect) 1991; 206: 297-300). Memantine binds to the PCP and MK-801 binding sites of the NMDA receptor in postmortem human frontal cortex at therapeutic concentrations (Kornhuber et al., Eur J Pharmacol 1989; 166: 589-590), and reduces membrane currents (Bormann, Eur J Pharmacol 1989; 66: 591-592).

Chemically, memantine (EBIXA™, NAMENDA™) is 1-amino-3,5-dimethyladamantane of the adamantane class.

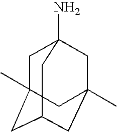

Memantine has a favorable pharmacological profile, is well tolerated and has been in clinical use for many years with minimal side-effects (Kornhuber et al., J Neural Transm Suppl 1994; 43: 91-104). Rarely has memantine been associated with significant side-effects such as cognitive defects, agitation, confusion, and psychosis (Rabey et al., J Neural Transm 1992; 4: 277-282; Riederer et al., Lancet, 1991 Oct. 19; 338(8773):1022-3) as seen with other NMDA antagonists, such as phencyclidine and ketamine. Memantine is well tolerated in the geriatric populations for which it is typically prescribed in Europe (Görtelmeyer et al., Arzneim-Forsch/Drug Res 1992; 42: 904-913).

Without being bound by theory, one possibility why memantine is less likely to induce cognitive deficits and psychosis may be due to its negligible effects on the hypothalamic-pituitary axis (HPA) compared to other NMDA antagonists such as ketamine. NMDA receptors have been reported to be involved in the physiologic pulsatile regulation of hormone release from the HPA axis (Bhat et al., Neuroendocrinology. 62(2): 187-97, 178-186 (1995)) resulting in hypercortisolemia. Psychotic symptoms and cognitive deficits in multiple sclerosis have been linked to an increased dopamine activity secondary to this HPA overactivity (Walder et al., Biol Psychiatry 2000; 48: 1121-1132). The lack of memantine's effect on the HPA axis and resulting increased dopamine activity may be an explanation for the low rates of psychosis seen with this drug.

Memantine has significant neurotrophic and modulatory properties, and it can be used to modulate glutamatergic neurotransmission, while also providing for robust neurotrophic effects via direct intracellular mechanisms. Memantine displays potent non-competitive voltage-dependent NMDA antagonist properties with effects comparable to MK-801 (see, Bormann, Eur J Pharmacol 1989; 66: 591-592). Memantine also demonstrates anticonvulsant and neuroprotective properties and dopaminergic effects in vitro (see, Maj, Arzneim Forsch/Drug Res 1982; 32: 1236-1273). Memantine has been used since 1978 and is approved in Germany for the treatment of mild and moderate cerebral performance disorders with the following cardinal symptoms: concentration and memory disorders, loss of interest and drive, premature fatigue, and dementia syndrome, as well as in diseases in which an increase of attention and alertness (vigilance) is required. Cerebral and spinal spasticity, Parkinson and Parkinson-like diseases are other indications for which memantine can be used.

In states of a reduced glutamate release, after degeneration of neurons, memantine results in an improvement in signal transmission and activation of neurons. In the state of a massive glutamate release, e.g., ischemia, memantine blocks NMDA receptors that mediate the excitotoxic action of glutamate on neurons. It is believed that its neuroprotective properties are due to NMDA receptor antagonism in pathologies with increased glutamate. Memantine's efficacy in Parkinson's disease has been suggested to be a result of its ability to neutralize (or modulate) the increased activity of the glutamatergic cortico-striatal and subthalamicopallidal pathways (Klockgether and Turski, Trends Neurosci 1989; 12: 285-286; Ann Neurol 1990; 28: 539-546, and Schmidt et al., Trends Neurosci 1990; 13: 46-47). This effect is independent of dopamine or norepinephrine release.

Memantine has been reported for many years to have positive effects on deficit symptoms or depressive symptoms commonly found in other neurological conditions such as Parkinson's disease and dementia. In studies of patients with dementia and Parkinson's disease, the symptoms of depressed mood, anxiety, lack of drive, somatic disturbances, impairment in vigilance, short-term memory and concentration were significantly improved with memantine. Some of these studies also reported the adverse events of hyperactivity, restlessness, and euphoria with memantine. Thus, memantine may have similar activating effects upon the symptoms of multiple sclerosis.

Another uncompetitive NMDA receptor channel antagonist which has been proven effective to treat a variety of afflictions, such as rimantadine (1-(1-aminoethyl)adamantane, FLUMADINE™), for the prophylaxis and treatment of influenza in humans.

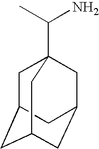

Amantadine (1-amino-adamantane, SYMMETREL™) has been used for the treatment of both influenza and Parkinson's disease (Schwab et al., J. Am. Med. Assoc. (1969) 208:1168).

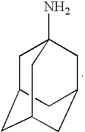

Pharmaceutical compositions comprising an uncompetitive NMDA receptor channel antagonist in an effective amount(s) to treat multiple sclerosis are also included in the invention. The methods described herein can be carried out either in vivo or in vitro (or ex vivo).

The uncompetitive NMDA receptor channel antagonist agents used in compositions of the invention are administered at a dosage of generally from 30-400 mg/day. For example, for memantine the dosage is preferably greater than 30 mg/day, e.g., about from about 30 to about 80 mg/day. Memantine is administered at 30, 40, 50, 60, 70, or 80 mg/day. Amantadine is administered from about 150 to about 400 mg/day, e.g., at 180, 200, 250, 300, 350, or 400 mg/day. Rimantadine is administered from about 150 to about 400 mg/day, e.g., at 180, 200, 250, 300, 350, or 400 mg/day. Memantine is particularly preferred. In a preferred embodiment, the compound of the invention is taken orally once a day or twice a day.

The present invention provides a more effective method of treatment for multiple sclerosis, and pharmaceutical compositions for treating multiple sclerosis, which may be used in such methods. In an embodiment, the invention relates to methods for treating a subject having multiple sclerosis, through the administration of a composition containing one or more uncompetitive NMDA receptor channel antagonists.

In one embodiment, methods of treating multiple sclerosis are disclosed, wherein an uncompetitive NMDA receptor channel antagonist is administered to a subject having multiple sclerosis such that the multiple sclerosis is treated or at least partially alleviated. The uncompetitive NMDA receptor channel antagonist is administered as part of a pharmaceutical composition. In another embodiment, a patient is diagnosed, e.g., to determine if treatment is necessary, whereupon a composition in accordance with the invention is administered to treat the patient. The amount of uncompetitive NMDA receptor channel antagonist is typically effective to reduce symptoms and to enable an observation of a reduction in symptoms.

Schedule of Administration

The compositions of the invention are administered in any suitable fashion to obtain the desired treatment of multiple sclerosis in the patient.

The present invention provides a more effective method of treatment for multiple sclerosis, and pharmaceutical compositions for treating multiple sclerosis, which may be used in such methods.

The invention further relates to kits for treating patients having multiple sclerosis, comprising a therapeutically effective dose of uncompetitive NMDA receptor channel antagonist for treating or at least partially alleviating the symptoms of the condition, and instructions for its use.

The present invention is suitable for the reduction of multiple sclerosis symptoms. Symptoms associated with, or arising from, multiple sclerosis, include fatigue, pain and tingling in the arms and legs; localized and generalized numbness, muscle spasm and weakness; bowel and bladder dysfunction; and difficulty with balance when walking or standing. The amount of uncompetitive NMDA receptor channel antagonist is typically effective to reduce symptoms and to enable an observation of a reduction in symptoms

To evaluate whether a patient is benefiting from the (treatment), one examines the patient's symptoms in a quantitative way, e.g., by decrease in the symptoms of motor dysfunction, improvement in cognitive abilities or reduction in decline of cognitive abilities, or in reduction in psychiatric symptomatology. In a successful treatment, the patient status will have improved (i.e., decrease in the symptoms, improvement in cognitive abilities or reduction in decline of cognitive abilities, or in reduction in psychiatric symptomatology).

As for every drug, the dosage is an important part of the success of the treatment and the health of the patient. In every case, in the specified range, the physician has to determine the best dosage for a given patient, according to his sex, age, weight, pathological state and other parameters.

The pharmaceutical compositions of the present invention contain a therapeutically effective amount of the active agents. The amount of the compound will depend on the patient being treated. The patient's weight, severity of illness, manner of administration and judgment of the prescribing physician should be taken into account in deciding the proper amount. The determination of a therapeutically effective amount of an uncompetitive NMDA receptor channel antagonist is well within the capabilities of one with skill in the art.

In some cases, it may be necessary to use dosages outside of the ranges stated in pharmaceutical packaging insert to treat a patient. Those cases will be apparent to the prescribing physician. Where it is necessary, a physician will also know how and when to interrupt, adjust or terminate treatment in conjunction with a response of a particular patient.

Formulation and Administration

The compounds of the present invention are administered in a suitably formulated dosage form. Compounds are administered to a patient in the form of a pharmaceutically acceptable salt or in a pharmaceutical composition. A compound that is administered in a pharmaceutical composition is mixed with a suitable carrier or excipient such that a therapeutically effective amount is present in the composition. The term “therapeutically effective amount” refers to an amount of the compound that is necessary to achieve a desired endpoint (e.g., decreasing symptoms associated with multiple sclerosis).

A variety of preparations can be used to formulate pharmaceutical compositions containing the uncompetitive NMDA receptor channel antagonists, including solid, semi solid, liquid and gaseous forms. Techniques for formulation and administration may be found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants and aerosols are examples of such formulations. The formulations can be administered in either a local or systemic manner or in a depot or sustained release fashion. Administration of the composition can be performed in a variety of ways. In a preferred embodiment, the route of administration is oral. In other embodiments, the route is buccal, rectal, parenteral, intraperitoneal, intradermal, transdermal, intranasal, and intratracheal means can be used. The compositions of the invention may be administered in combination with a variety of pharmaceutical excipients, including stabilizing agents, carriers and/or encapsulation formulations as described herein.

The preparation of pharmaceutical or pharmacological compositions will be known to those of skill in the art in light of the present disclosure. Typically, such compositions may be prepared as solid forms; as tablets or other solids for oral administration; as time release capsules.

For human administration, preparations should meet sterility CMC manufacturing standards as required by FDA.

Administration of compounds are anticipated to be oral delivery (solid or liquid). A particularly convenient frequency for the administration of the compounds of the invention is once a day or twice a day.

Upon formulation, therapeutics will be administered in a manner compatible with the dosage formulation, and in such amount as is pharmacologically effective. The formulations are easily administered in a variety of dosage forms, such as oral formulations described, but modified drug release tablets and capsules and the like can also be employed. In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically used. Suitable regimes for administration are also variable, but would be typified by initially administering the compound and monitoring the results and then giving further controlled doses at further intervals. The compounds of the invention can be formulated by dissolving, suspending or emulsifying in an aqueous or nonaqueous solvent. Vegetable (e.g., sesame oil) or similar oils, synthetic aliphatic acid glycerides, esters of higher aliphatic acids and propylene glycol are examples of nonaqueous solvents. Aqueous solutions such as Hank's solution, Ringer's solution or physiological saline buffer can also be used.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Oral preparations can be formulated through combination with pharmaceutically acceptable carriers that are well known in the art. The carriers enable the compound to be formulated, for example, as a tablet, pill, capsule, solution, suspension, sustained release formulation; powder, liquid or gel for oral ingestion by the patient. Oral use formulations can be obtained in a variety of ways, including mixing the compound with a solid excipient, optionally grinding the resulting mixture, adding suitable auxiliaries and processing the granule mixture. The following list includes examples of excipients that can be used in an oral formulation: sugars such as lactose, sucrose, mannitol or sorbitol; cellulose preparations such as maize starch, non gluten wheat starch, potato starch, gelatin, gum tragacanth, methyl cellulose, hydroxypropylmethylcellulose, sodium carboxymethylcellulose and polyvinylpyrrolidone (PVP). Oral formulations include such normally employed excipients as, for example, pharmaceutical grades of mannitol, lactose, starch, magnesium stearate, sodium saccharine, cellulose, magnesium carbonate and the like.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

The compositions of the present invention can also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage can be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The subject treated by the methods of the invention is a mammal, more preferably a human. The following properties or applications of these methods will essentially be described for humans although they may also be applied to non-human mammals, e.g., apes, monkeys, dogs, mice, etc. The invention therefore can also be used in a veterinarian context.

The pharmaceutical compositions of the invention are used to treat multiple sclerosis. Also treated by the pharmaceutical compositions of the invention are symptoms arising from multiple sclerosis. Symptoms associated with, or arising from, multiple sclerosis, include movement disorders, such as involuntary movements, abnormal movements, and chorea; cognitive changes, such as intellectual deterioration, difficulties in mental flexibility, difficulty learning new information, and difficulty in memory recall; and psychiatric symptoms, such as depression, anxiety, obsessiveness, irritability, impulsiveness, social withdrawal, difficulty initiating activity, psychosis, hallucinations, delusions, and suicidality.

### EXAMPLES

### Example 1

### Memantine Trials

In this example, a series of comparative studies of memantine dosages for multiple sclerosis is described. The study is a multi-centre, double-blind, randomized, placebo-controlled efficacy study of various doses of memantine. The trial enrols 125 patients with MS at 6-10 sites. Study duration is 1 year.

Patients.

Patients eligible for this study include IFN-naive patients, between the ages of 18-55, diagnosed within the past 2 years with relapsing-remitting MS (RR-MS). Such patients will typically have evidence of demyelination on MRI scanning of the brain and have an Extended Disability Status Scale (EDSS) score between 0 and 3.5.

Study Design.

Treatment, Double-Blind, Efficacy Study.

Study Assessments.

The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Treatment.

Patients are randomized to receive one of the following study arms: Arm 1: memantine 30, mg oral daily; Arm 2: memantine 40 mg/day; Arm 3: memantine 50 mg/day; Arm 4: memantine 60 mg/day; Arm 5: memantine 70 mg/day; Arm 6: memantine 80 mg/day; Arm 7, placebo. The study lasts a total of 1 year.

### Example 2

### Amantadine Trails

In this example, a series of comparative studies of memantine dosages for multiple sclerosis is described. The study is a multi-centre, double-blind, randomized, placebo-controlled efficacy study of various doses of memantine. The trial enrols 125 patients with MS at 6-10 sites. Study duration is 1 year.

Patients.

Patients eligible for this study include IFN-naive patients, between the ages of 18-55, diagnosed within the past 2 years with relapsing-remitting MS (RR-MS). Such patients will typically have evidence of demyelination on MRI scanning of the brain and have an Extended Disability Status Scale (EDSS) score between 0 and 3.5.

Study Design.

Treatment, Double-Blind, Efficacy Study.

Study Assessments.

The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Treatment.

Patients are randomized to receive one of the following study arms: Arm 1: amantadine 180, mg oral daily; Arm 2: amantadine 200 mg/day; Arm 3: amantadine 250 mg/day; Arm 4: amantadine 300 mg/day; Arm 5: amantadine 350 mg/day; Arm 6: amantadine 400 mg/day; Arm 7, placebo. The study lasts a total of 1 year.

### Example 3

### Rimantadine Trials

In this example, a series of comparative studies of memantine dosages for multiple sclerosis is described. The study is a multi-centre, double-blind, randomized, placebo-controlled efficacy study of various doses of memantine. The trial enrols 125 patients with MS at 6-10 sites. Study duration is 1 year.

Patients.

Patients eligible for this study include IFN-naive patients, between the ages of 18-55, diagnosed within the past 2 years with relapsing-remitting MS (RR-MS). Such patients will typically have evidence of demyelination on MRI scanning of the brain and have an Extended Disability Status Scale (EDSS) score between 0 and 3.5.

Study Design.

Treatment, Double-Blind, Efficacy Study.

Study Assessments.

The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Treatment.

Patients are randomized to receive one of the following study arms: Arm 1: rimantadine 180, mg oral daily; Arm 2: rimantadine 200 mg/day; Arm 3: rimantadine 250 mg/day; Arm 4: rimantadine 300 mg/day; Arm 5: rimantadine 350 mg/day; Arm 6: rimantadine 400 mg/day; Arm 7, placebo. The study lasts a total of 1 year.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby fully incorporated by reference. The appropriate components, processes, and methods of those patents, applications and other documents may be selected for the present invention and embodiments thereof.

1. A method of treating multiple sclerosis comprising administering to a subject having multiple sclerosis an uncompetitive NMDA receptor channel antagonist, such that said multiple sclerosis is treated or at least partially alleviated.

2. A method of treating multiple sclerosis, comprising administering to a patient in need thereof a pharmaceutical composition comprising an uncompetitive NMDA receptor channel antagonist, in an amount effective to treat said multiple sclerosis in said patient.

3. A method of treating multiple sclerosis, comprising diagnosing a patient in need of treatment and administering to a patient in need thereof an uncompetitive NMDA receptor channel antagonist, such that said multiple sclerosis is treated or at least partially alleviated.

4. The method of claim 1, wherein the amount of said uncompetitive NMDA receptor channel antagonist is effective to reduce symptoms and to enable an observation of a reduction in symptoms.

5. The method of claim 1, wherein said uncompetitive NMDA receptor channel antagonist is selected from the group consisting of memantine, rimantadine, and amantadine.

6. The method of claim 1, wherein said treatment is administered orally.

7. The method of claim 1, wherein the amount of said uncompetitive NMDA receptor channel antagonist is at least about 30 to 400 mg per day.

8. The method of claim 1, wherein the dose of uncompetitive NMDA receptor channel antagonist is at least about 30 to 180 mg per day.

9. The method of claim 1, wherein the dose of uncompetitive NMDA receptor channel antagonist is at least about 30 to 80 mg per day.

10. The method of claim 1, wherein the dose of uncompetitive NMDA receptor channel antagonist is at least about 180 to 400 mg per day.

11. A kit for treating a patient having multiple sclerosis, comprising a therapeutically effective dose of an uncompetitive NMDA receptor channel antagonist, and instructions for its use.

12. The kit of claim 11 wherein said uncompetitive NMDA receptor channel antagonist is selected from the group consisting of memantine, rimantadine, and amantadine.

13. A pharmaceutical composition comprising an uncompetitive NMDA receptor channel antagonist, in an effective amount to treat multiple sclerosis.

14. The pharmaceutical composition according to claim 13, wherein the uncompetitive NMDA receptor channel antagonist is selected from the group consisting of memantine, rimantadine, and amantadine.

15. A method for treating multiple sclerosis comprising administering to a subject having a symptom of multiple sclerosis an uncompetitive NMDA receptor channel antagonist in an amount to effective to reduce the symptom and to enable an observation of a reduction in the symptom.

16. The method of claim 15, wherein the symptom is selected from the group consisting of fatigue, pain and tingling in the arms and legs; localized numbness, generalized numbness, muscle spasm, muscle weakness; bowel dysfunction, bladder dysfunction; and difficulty with balance when walking or standing.

17. A method of treating multiple sclerosis comprising administering to a subject having multiple sclerosis memantine at a dosage of at least 30 mg/day.
